(19) United States
(12) Patent Application Publication
(10) Pub. No.: US 2007/0066572 A1
(43) Pub. Date: Mar. 22, 2007

(54) Neuroprotection by positively-charged nitroxides

(76) Inventors: Kalyanaraman Balaraman, Wauwatosa, WI (US); Joy Joseph, New Berlin, WI (US); Rugao Liu, Grand Forks, ND (US); Liying Chi, Grand Forks, ND (US)

Correspondence Address: QUARLES & BRADY LLP, 411 E. WISCONSIN AVENUE, SUITE 2040, MILWAUKEE, WI, 53202-4497, US

(21) Appl. No.: 11/520,191

(22) Filed: Sep. 13, 2006

Related U.S. Application Data
(60) Provisional application No. 60/716,914, filed on Sep. 14, 2005.

(51) Int. Cl. A61K 31/675

(52) U.S. Cl. 514/89

(57) ABSTRACT

Methods for treating neurodegenerative disorders characterized by abnormal reactive oxygen species including administering to a subject an effective amount of a mitochondria-targeted nitroxide. Likewise, methods for mitigating reactive-oxygen species-mediated apoptosis including administering an effective amount of a mitochondria-targeted nitroxide.

### CROSS-REFERENCE TO RELATED APPLICATION

This application claims the benefit of U.S. Provisional Patent Application No. 60/716,914, filed Sep. 14, 2005, incorporated herein by reference as if set forth in its entirety.

### STATEMENT REGARDING FEDERALLY SPONSORED RESEARCH OR DEVELOPMENT

This invention was made with United States government support awarded by NIH Grant No. HL63119. The United States government has certain rights in this invention.

### BACKGROUND

The invention relates generally to methods for attenuating the effects of reactive oxygen species, and more particularly to administering a mitochondria-targeted antioxidant to attenuate the effects of reactive oxygen species in a subject having or at risk for a neurodegenerative disorder such as amyotrophic lateral sclerosis (ALS).

The mitochondrial electron transport chain is a source of reactive oxygen species (ROS; e.g. superoxide (O₂′⁻) and hydrogen peroxide (H₂O₂)). Accordingly, mitochondria are more vulnerable to oxidative damage than other cellular organelles.

Two major sites of O₂′⁻ generation in the mitochondrial electron transport chain are complexes I and III. O₂′⁻ generated within mitochondria activates uncoupling proteins through formation of lipid peroxidation breakdown products (e.g., 4-hydroxynonenol). Typically, mitochondrial O₂′⁻ is dismutated by manganese superoxide dismutase (MnSOD) that is localized within the mitochondrial matrix. O₂′⁻ can also react with nitric oxide (NO) at a diffusion-controlled reaction rate, forming a highly potent oxidant, peroxynitrite (ONOO⁻) that can modify proteins and DNA through oxidation and nitration reactions. Therefore, it is essential to abrogate O₂′⁻—induced redox signaling in mitochondria to prevent mitochondrial dysfunction.

Generally, mitochondria are well-equipped to detoxify other ROS, such as H₂O₂, due to the presence of antioxidant enzymes (e.g., peroxiredoxins, thioredoxins and GSH-dependent peroxidases).

Mitochondrial dysfunction is a crucial factor in regulating apoptotic cell death in various neurological and age-related disorders such as ALS, Parkinson's, Huntington's and Alzheimer's diseases, as well as Friedreich ataxia. Other relevant mitochondrial disorders include diabetes, ischemia and reperfusion injury, and radiation-induced mitochondrial dysfunction. In these disorders, ROS-scavenging systems either fail or are overwhelmed. For example, increased formation of ROS leads to mitochondrial dysfunction and cell death in many neurodegenerative disorders. Specifically, ROS impair mitochondrial ATP synthesis and calcium homeostasis, leading to mitochondrial dysfunction.

Of particular interest herein is ALS, a paralytic disorder characterized by degeneration of large motor neurons of the brain, the spinal cord and the cerebral cortex. Approximately, 10% of ALS cases are genetic, with the remainder being sporadic. Rosen D, “Mutations in Cu/Zn superoxide dismutase gene are associated with familial amyotrophic lateral sclerosis,” Nature 362:59-62 (1993); and Brown R, “Superoxide dismutase and familial amyotrophic lateral sclerosis: new insights into mechanisms and treatments,” Ann. Neurol. 39:145-146 (1996).

The genetic form of ALS is associated with mutations in superoxide dismutase (SOD1), an enzyme that scavenges O₂′⁻ and converts it to less reactive species. Said M, et al., “Increased reactive oxygen species in familial amyotrophic lateral sclerosis with mutations in SOD1,” J. Neurol. Sci. 176:88-94 (2000). Currently, over 100 SOD1 mutations at 26 different amino acids have been identified. Rosen D, et al., “Genetic linkage analysis of familial amyotrophic lateral sclerosis using human chromosome 21 microsatellite DNA markers,” Am. J. Med. Genet. 51:61-69 (1994). One frequently observed mutation is a G93A—glycine to alanine mutation at amino acid 93. In fact, mice transfected with human SOD1 and human SOD1-G93A plasmids are used as an animal model in ALS research. Gurney M, et al., “Benefit of vitamin E, riluzole, and gabapentin in a transgenic model of familial amyotrophic lateral sclerosis,” Ann. Neurol. 39:147-157 (1996); and Price D. et al., “Motor neurone disease and animal models,” Neurobiol. Dis. 1:3-11 (1994).

Antioxidants (e.g., α-tocopherol, ubiquinol and N-acetylcysteine) can decrease mitochondrial oxidative damage. Kagan V, et al., “Assay of ubiquinones ubiquinols as antioxidants,” Methods Enzymol. 234:343-354 (1994); Maguire J, et al., “Mitochondrial electron transport-linked tocopheroxyl radical reduction,” J. Biol. Chem. 264:21462-21465 (1989); and Ernster L, et al., “The mode of action of lipid-soluble antioxidants in biological membranes: relationship between the effects of ubiquinol and vitamin E as inhibitors of lipid peroxidation in submitochondrial particles,” Biofactors. 3:241-248 (1992). Unfortunately, these antioxidants do not sufficiently accumulate in mitochondria, thereby limiting their effectiveness in clinical applications.

Murphy et al., reported that antioxidants covalently coupled to a lipophilic triphenylphosphonium cation were preferentially taken up by mitochondria. Murphy M & Smith R, “Drug delivery to mitochondria: the key to mitochondrial medicine,” Adv. Drug Deliv. Rev. 41:235-250 (2000); and Murphy M, “Selective targeting of bioactive compounds to mitochondria,” Trends Biotechnol. 15:326-330 (1997). The lipophilic triphenylphosphonium cation easily permeates through lipid bilayers and subsequently accumulates in mitochondria because of its large membrane potential.

Nitroxides (also called amine oxides) are organic molecules having a nitroxyl free radical and have been shown to be stable inhibitors of ROS-induced damage. Nitroxides inhibit oxidative damage by scavenging free radicals, by reducing hypervalent species and by mimicking SOD. Chang T, “Peroxiredoxin III, a mitochondrion-specific peroxidase, regulates apoptotic signaling by mitochondria,” J. Biol. Chem. 279:41975-41984 (2004). Nitroxides, such as Tempone (2,2,6,6-tetramethyl-4-piperidone-1-oxyl), undergo a one-electron reduction in the presence of O₂′⁻, forming a hydroxylamine and oxygen (O₂). The hydroxylamine can be oxidized back to the original nitroxide and H₂O₂. As shown in Table 1, the net effect is dismutation of two molecules of O₂′⁻ to H₂O₂ and O₂. Like many other antioxidants, nitroxides do not accumulate in any significant concentration in mitochondria.

TABLE 1
Dismutation of Superoxide by Tempone
Tempone + O₂⁻ → Tempone Hydroxylamine + O₂
Tempone Hydroxylamine + O₂⁻ → Tempone + H₂O₂
(Tempone)
O₂⁻ + O₂⁻ + 2H⁺ → H₂O₂ + O₂

Accordingly, there is a need for methods of protecting cells from the effects of ROS-induced damage with mitrochondria-targeted nitroxides.

### BRIEF SUMMARY

In a first aspect, a method of treating amyotrophic lateral sclerosis includes administering to a subject having or at risk of having amyotrophic lateral sclerosis an effective amount of a mitochondria-targeted nitroxide. The mitochondria-targeted nitroxide can be a positively charged nitroxide ether, a positively charged nitroxide ester or a positively charged nitroxide amide. The mitochondria-targeted nitroxide attenuates the symptoms of the neurodegenerative disorder are attenuated.

In some embodiments, the mitochondria-targeted nitroxide can be chosen from Mito-Tempol ether, Mito-Tempol ester, Mito-Tempol amide, Mito-CP, Mito-Tempone, Mito-Tempamide, Mito-Proxyl ether, Mito-Proxyl ester, Mito-Proxyl amide, Tributylalkylammonium Tempol ether and Tribenzyalkylammonium Tempol ether. In other embodiments, the mitochondria-targeted nitroxide is Mito-Tempol ether. In other embodiments, the mitochondria-targeted nitroxide is Mito-Tempol ester.

In a second aspect, a method of attenuating mitochondria dysfunction includes administering to a subject at having or at risk of having reactive oxygen species-mediated mitochondria dysfunction an effective amount of a mitochondria-targeted nitroxide such that mitochondria dysfunction is attenuated. The mitochondria-targeted nitroxide can be a positively charged nitroxide ether, a positively charged nitroxide ester or a positively charged nitroxide amide. The mitochondria-targeted nitroxide attenuates apoptosis.

In some embodiments, the mitochondria-targeted nitroxide can be chosen from Mito-Tempol ether, Mito-Tempol ester, Mito-Tempol amide, Mito-CP, Mito-Tempone, Mito-Tempamide, Mito-Proxyl ether, Mito-Proxyl ester, Mito-Proxyl amide, Tributylalkylammonium Tempol ether and Tribenzyalkylammonium Tempol ether. In other embodiments, the mitochondria-targeted nitroxide is Mito-Tempol ether. In other embodiments, the mitochondria-targeted nitroxide is Mito-Tempol ester.

In some embodiments, the reactive oxygen species-mediated mitochondria dysfunction is caused by diabetes, hypertension, ischemia and reperfusion injury, or radiation-induced mitochondria dysfunction.

In a third aspect, a method of preparing Mito-Tempol includes a first step of obtaining a sodium derivative of Tempol. The method includes a second step of obtaining a bromobutyl ether of Tempol from the sodium derivative of Tempol. The method includes a third step of combining the bromobutyl ether of Tempol with triphenylphosphine to obtain Mito-Tempol. The method includes a fourth step of analyzing purity of the Mito-Tempol with electron paramagnetic resonance and liquid chromatography-mass spectrometry such that the purity is at least 90%. The purity, however, can also be at least 95% or alternatively at least 99%.

While the invention defined by the appended claims is susceptible to various modifications and alternative forms, specific embodiments thereof have been shown by way of example in the drawings and are herein described in detail. It should be understood, however, that the description of specific embodiments is not intended to limit the invention to the particular forms disclosed, but on the contrary, the intention is to cover all modifications, equivalents and alternatives falling within the spirit and scope of the invention as defined by the appended claims.

### BRIEF DESCRIPTION OF THE DRAWINGS

The invention will be better understood when consideration is given to the following detailed description thereof. Such detailed description makes reference to the following drawings, wherein:

FIG. 1 shows a positively charged Tempol ether;

FIG. 2 shows that Mito-Tempol delays ALS disease progression in ALS-like mice;

FIG. 3 shows that Mito-Tempol extends ALS-like mice lifespan;

FIG. 4 shows that Mito-Tempol promotes motor function during disease onset and progression;

FIG. 5 shows that Mito-Tempol attenuates caspase-3 activity; and

FIG. 6 shows chemical structures of mitochondria-targeted nitroxides.

### DESCRIPTION OF PREFERRED EMBODIMENTS

The present invention relates to our observation that mitochondria-targeted nitroxides are effective inhibitors of ROS-induced damage seen in some neurodegenerative disorders.

Unless defined otherwise, all technical and scientific terms used herein have the same meaning as commonly understood by one of ordinary skill in the art to which the invention belongs. Although any methods and materials similar to or equivalent to those described herein can be used in the practice or testing of the present invention, the preferred methods and materials are now described.

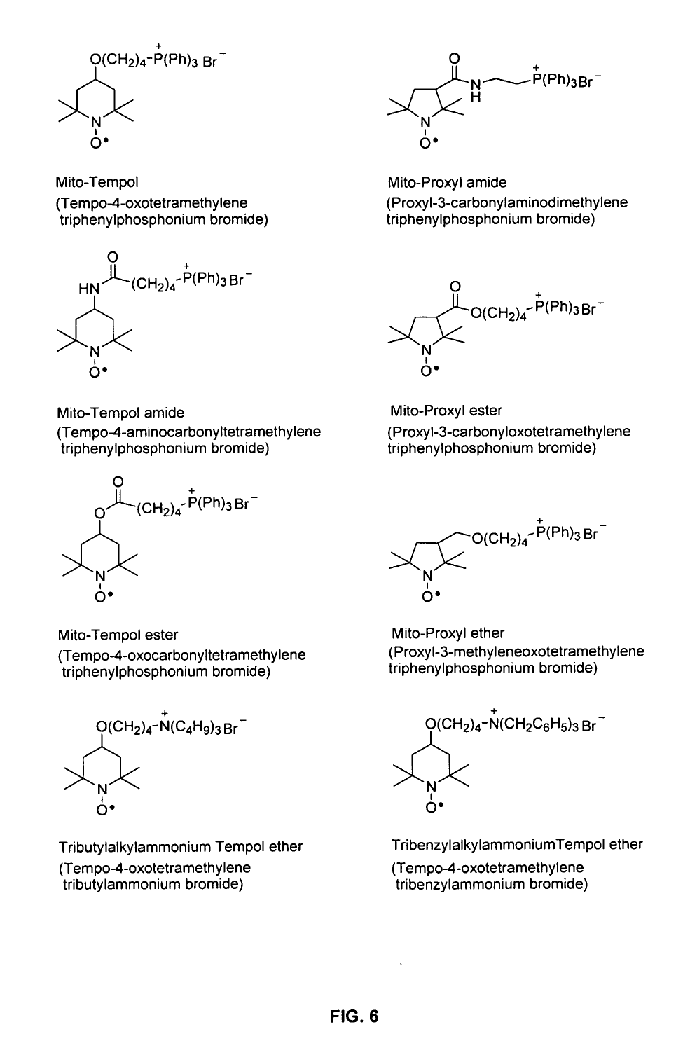

As used herein, a nitroxide is any five-membered or six-membered ring having a stabilized nitroxide moiety. Additionally, the ring may have an ether, an ester or an amide attached thereto. FIG. 6 shows non-limiting examples of nitroxides with varying alkyl chain lengths, such varied chain lengths may be used to increase or decrease hydrophobicity.

As used herein, a mitochondria-targeted nitroxide refers to any nitroxide conjugated to a molecule that increases its lipid bilayer permeability. Also as shown in FIG. 6, such molecules include, but are not limited to, a triphenylphosphonium group or a benzyl ammonium group.

As used herein, symptoms of a neurodegenerative disorder include, but are not limited to impaired motor neuron function, mitochondria dysfunction (i.e. impaired mitochondrial ATP synthesis and altered calcium homeostasis), increased ROS, oxidative damage, protein nitration, aggregation of proteins and decreased cell survival.

We hypothesize that mitochondria-targeted nitroxides may constitute a new class of targeted MnSOD mimetics. Using an untargeted nitroxide carboxy proxyl (CP) and a mitochondria-targeted carboxy proxyl (Mito-CP), we recently demonstrated that Mito-CP is far more effective in inhibiting peroxide-induced oxidative damage and apoptosis in endothelial cells. Dhanasekaran A, et al., “Mitochondria superoxide dismutase mimetic inhibits peroxide-induced oxidative damage and apoptosis: role of mitochondrial superoxide,” Free Radic. Biol. Med. 39:567-583 (2005), incorporated herein by reference as if set forth in its entirety. These results implicate a therapeutic use of mitochondria-targeted nitroxides as neuroprotective agents in neurodegenerative disorders, such as ALS, where ROS have been proposed to a be a major culprit.

Several lines of evidence suggest that ALS is a free radical disease involving mitochondrial energy failure. Increased oxidative damage, protein nitration, apoptosis and aggregation of proteins are characteristic hallmarks of ALS. Numerous agents with a potential antioxidant mechanism of action have been tried in ALS mice models; however, none of these agents had been targeted to mitochondria.

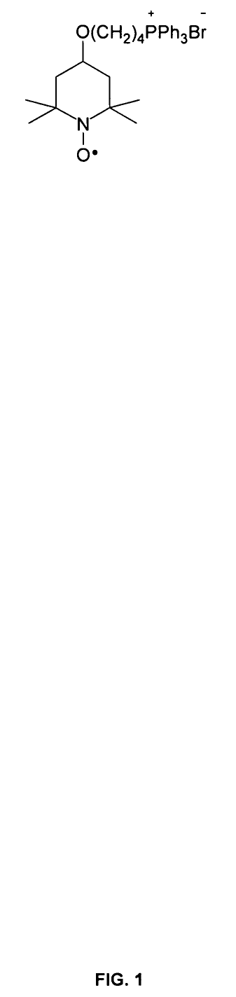

As the nitroxide, 4-hydroxy-2,2,6,6-tetramethylpiperidin-N-oxide (Tempol) has been used as an in vivo antioxidant in many diseases including hypertension, we coupled Tempol to the triphenylphsophonium cation and synthesized a positively-charged Tempol ether (FIG. 1). Although this particular compound was previously claimed to have been made (Dessolin J, et al., “Selective targeting of synthetic antioxidants to mitochondria: towards a mitochondrial medicine for neurodegenerative diseases?,” Eur. J. Pharm. 447:155-161 (2002)), when we synthesized this compound by following the published method the resulting product contained less than 1% of the nitoxide as measured by electron paramagnetic resonance (EPR). Although previous investigators have used other analytical procedures (e.g., nuclear magnetic resonance and mass spectroscopy), the most specific method to detect and characterize nitroxides is EPR. Therefore, as described below, we modified the published method for Mito-Tempol. EPR analysis of Mito-Tempol prepared by our modified method contained a stoichiometric amount of the nitroxide group. In addition, Mito-Tempol prepared by our method inhibited endothelial oxidative damage and apoptosis. Thus, EPR analysis of the nitroxide product establishes its structural integrity.

In a first aspect, a method of treating subjects having or at risk of having ALS includes administering an effective amount of a mitochondria-targeted nitroxide to a subject having or susceptible to having ALS so that the symptoms of ALS are attenuated preferably by at least about 10%, alternatively by at least about 15%, alternatively by at least about 20% and alternatively by at least about 25%.

In one embodiment of this aspect, the mitochondria-targeted nitroxide may be a positively charged nitroxide ether, a positively charged nitroxide ester or a positively charged nitroxide amide. In other embodiments, the mitochondria-targeted nitroxide may be Mito-Tempol ether, Mito-Tempol ester, Mito-Tempol amide, Mito-CP, Mito-Tempone, Mito-Tempamide, Mito-Proxyl ether, Mito-Proxyl ester, Mito-Proxyl amide, Tributylalkylammonium Tempol ether, or Tribenzyalkylammonium Tempol ether.

These compounds can be administered orally, as they are stable in drinking water. Alternatively, these compounds may be administered intravenously or intraperitoneally.

Mitochondria-targeted nitroxides are stable in aqueous solutions at a pH of about 7.0. Pharmaceutically acceptable carriers for mitochondria-targeted nitroxides may include chemicals such as beta-cyclodextrin.

Dosages suitable for this embodiment are in a range from about 30 mg/kg to about 60 mg/kg body weight of a mitochondria-targeted nitroxide at least daily. However, it is envisioned that the dosage and treatment with mitochondria-targeted nitroxides will differ for different subjects. It is noted that the number of doses a subject receives, the time allowed between doses and the length of time a subject receives mitochondria-targeted nitroxides will generally depend on the severity of the neurodegenerative disorder.

For example, Mito-Tempol may be given to a subject intravenously starting at least about 1 mg/kg to about 4 mg/kg at one to two times a week. This dosage level and the time between doses may be modified based on a physician's assessment of the disease progression.

In a second aspect, a method of attenuating mitochondria dysfunction includes administering to a subject at having or at risk of having reactive oxygen species-mediated mitochondria dysfunction an effective amount of a mitochondria-targeted nitroxide such that apoptosis is attenuated by at least about 10%, alternatively by at least about 15%, alternatively by at least about 20% and alternatively by at least about 25%.

In one embodiment of this aspect, the mitochondria-targeted nitroxide may be a positively charged nitroxide ether, a positively charged nitroxide ester or a positively charged nitroxide amide. In other embodiments, the mitochondria-targeted nitroxide may be Mito-Tempol ether, Mito-Tempol ester, Mito-Tempol amide, Mito-CP, Mito-Tempone, Mito-Tempamide, Mito-Proxyl ether, Mito-Proxyl ester, Mito-Proxyl amide, Tributylalkylammonium Tempol ether, or Tribenzyalkylammonium Tempol ether.

These compounds can be administered orally, as they are stable in drinking water. Alternatively, these compounds may be administered intravenously or intraperitoneally.

Mitochondria-targeted nitroxides are stable in aqueous solutions at a pH of about 7.0. Pharmaceutically acceptable carriers for mitochondria-targeted nitroxides may include chemicals such as beta-cyclodextrin.

Dosages suitable for this embodiment include about 30 mg/kg to about 60 mg/kg body weight of a mitochondria-targeted nitroxide at least daily. However, it is envisioned that the dosage and treatment with mitochondria-targeted nitroxides will differ for different subjects. It is noted that the number of doses a subject receives, the time allowed between doses and the length of time a subject receives mitochondria-targeted nitroxides will generally depend on the severity of the apoptosis.

For example, Mito-Tempol may be given to a subject intravenously starting at least about 1 mg/kg to about 4 mg/kg at one to two times a week. This dosage level and the time between doses may be modified based on a physician's assessment of the disease progression.

In a third aspect, a method of preparing Mito-Tempol that is at least 90% pure, alternatively at least 95% pure and alteratively at least 99% pure. The method includes obtaining a sodium derivative of Tempol, eventually a bromobutyl ether of Tempol is obtained. The bromobutyl ether of Tempol is combined with triphenylphosphine to obtain Mito-Tempol. The purity of the Mito-Tempol is assayed with electron paramagnetic resonance and liquid chromatography-mass spectrometry.

The invention will be more fully understood upon consideration of the following non-limiting Examples.

### EXAMPLES

### Example 1

### Synthesis of Mito-Tempol

Methods: Previous investigators (Dessolin et al., supra) used a large excess of a reductant (sodium hydride) during their attempted synthesis of Mito-Tempol. In addition, they used dimethylformamide as a solvent. This particular solvent is typically not used for nitroxide ether synthesis. However, we discovered two differences that led to an increased production of Mito-Tempol. A first difference in our method of synthesizing Mito-Tempol is that we used a stoichiometric amount of sodium hydride (i.e., the ratio of Tempol to sodium hydride is nearly 1:1). A suitable range is 1:1 to 1:2. A second difference in our method of synthesizing Mito-Tempol is that we used benzene as a solvent. One can also use other solvents, such as dioxane, toluene or heptane. We believe that these two differences are instrumental for our successful synthesis of Mito-Tempol.

Mito-Tempol was synthesized as follows (shown in Table 2): Tempol (3.44 g. or 20 mmole; Sigma-Aldrich Chemical Co.; Milwaukee, Wis.) was added to a 3-neck (250 ml) flask containing dry benzene (100 ml; Sigma-Aldrich). The flask was evacuated and filled with dry nitrogen (Sigma-Aldrich) three times. Sodium hydride (0.72 g or 30 mmole; Sigma-Aldrich) was added to the flask, under stirring, and refluxed for 24 hours. After cooling the flask in ice-cold water, 1,4-dibromobutane (8.6 g or 40 mmole; Sigma-Aldrich) was added and refluxed under nitrogen for 24 hours. The flask was again cooled in ice-cold water and 50 ml of cold water was added to the flask. The contents were transferred to a separatory funnel and gently shaken. A colored upper layer was collected, dried over magnesium sulfate (Sigma-Aldrich) and solvent removed by rotary evaporation. The residual oil was purified by column chromatography on a Silica gel-60 (Sigma-Aldrich). Unreacted dibromobutane was eluted with hexane (Sigma-Aldrich). The desired product (a colored band) was eluted with an ether/hexane (1:1) solvent mixture. An orange oil (5 g, 82% yield) was obtained by removing the solvent from the relevant fractions. Thin-layer chromatography in hexane/ether (1:1 mixture) using a Silica gel-60 was used to verify the purity of the product (one spot at an Rf of 0.75).

The bromobutyl ether of Tempol (5 g) was added to a 200 ml flask containing 50 ml dioxane (Sigma-Aldrich) and 8 g triphenylphosphine (Sigma-Aldrich). The flask was partially evacuated and filled with dry nitrogen three times. The contents were heated again under reflux for 24 hours and further cooled down in an ice bath. The solvent was removed in a rotary evaporator to obtain a syrupy liquid. The reaction mixture was added to ether (200 ml; Sigma-Aldrich), stirred and decanted off the solvent. The precipitate was dissolved in dichloromethane (20 ml; Sigma-Aldrich) and added to ether (100 ml). The resulting precipitate was collected by decantation and further purified by dissolving in dichloromethane and precipitating by adding ether. This procedure was repeated three times. The final residue was dried on vacuum to get a fluffy brown solid that was hygroscopic. The purity of the final product was ascertained by HPLC using a C-16 column, LC-MS (mass weight, 489.5), and by EPR spectroscopy. The overall yield was 6.5 g (60%).

TABLE 2
Synthesis of Mito-Tempol

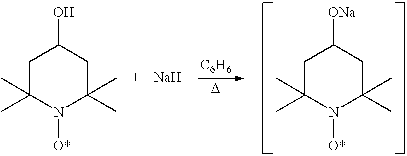

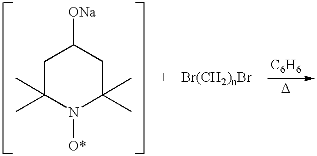

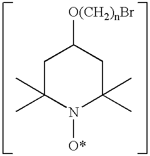

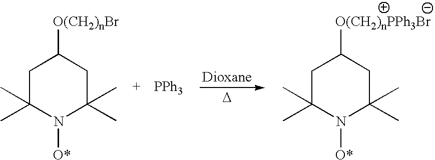

Using the method of Dessolin et al., supra, we obtained a product that had a nitroxide concentration of about 1-2% as measured by EPR. A major difference in the experimental design between our procedure and that of Dessolin et al., is the ratio of Tempol to sodium hydride. Dessolin et al. used a 6 mole equivalent of sodium hydride as opposed to 1.5-2.0 equivalent of sodium hydride used in our procedure. As a result of the large excess of sodium hydride, it is plausible that Tempol nitroxide was reduced to the corresponding hydroxylamine, which then coupled with the dibromobutane to form a hydroxylamino ether in dimethylformamide solvent used in that study. The resulting product would have the same NMR and mass ion as that of Mito-Tempol. However, Dessolin et al., did not use EPR to confirm the nitroxide structure. Thus, the claims that Dessolin et al. have synthesized Mito-Tempol are unsubstantiated.

In addition, we prepared other mitochondria-targeted nitroxides having Tempol. Tables 3 and 4 show synthesis methods for a Mito-Tempol ester (starting material is Tempol) and Mito-tempamide (starting material is 4-amino-TEMPO (4-amino-2,2,6,6-tetramethylpiperidine-1-oxyl)).

TABLE 3
Synthesis of Mito-Tempol Ester

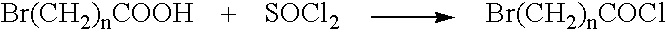

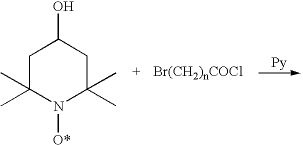

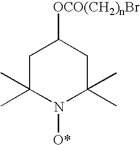

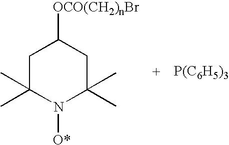

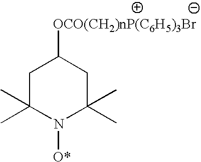

TABLE 4
Synthesis of Mito-Tempamide

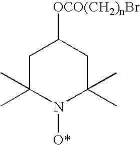

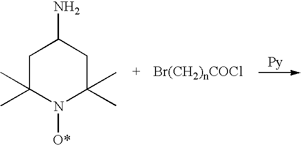

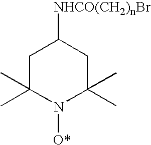

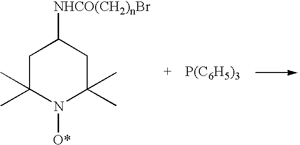

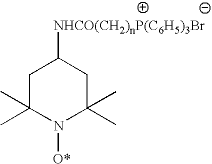

### Example 2

### In Vivo Attenuation of ALS Symptoms

Methods: Mito-Tempol was prepared as described above.

Animal model: A transgenic mouse line overexpressing mutant G93A-SOD1 gene (TgN(SOD1-G93A)1Gur, herein called hSOD1-G93A, a high expression mouse line) and the mating pairs were purchased from Jackson Laboratory (Bar Harbor, Me.). Mice were housed and bred as described previously (Liu R, et al., “Enhanced oxygen radical production in a transgenic mouse model of familial amyotrophic lateral sclerosis,” Ann. Neurol. 44:763-70 (1998)) and in accordance with the University of North Dakota Animal Care and Use Committee guidelines.

At age 40-50 days, transgenic littermates (wild-type normal mice and hSOD1-G93A mice) were divided into three groups (n=10) and administered with vehicle alone (regular drinking water), Mito-Tempol (40 mg/kg or about 1 mg/mouse/day) or Tempol (40 mg/kg or about 1 mg/mouse/day), respectively. The onset of disease was defined as the day when a sharp decrease in muscle strength was detected. The initial symptoms of hind leg paralysis was considered as the disease progression as well.

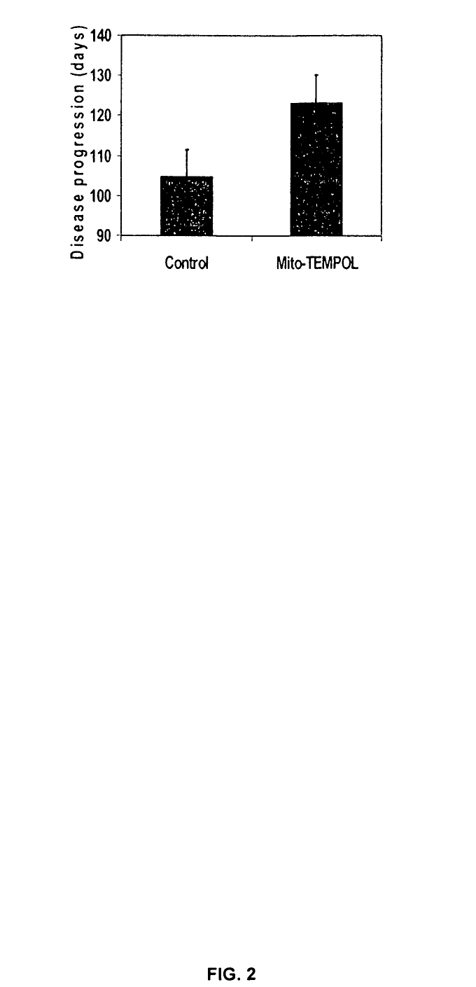

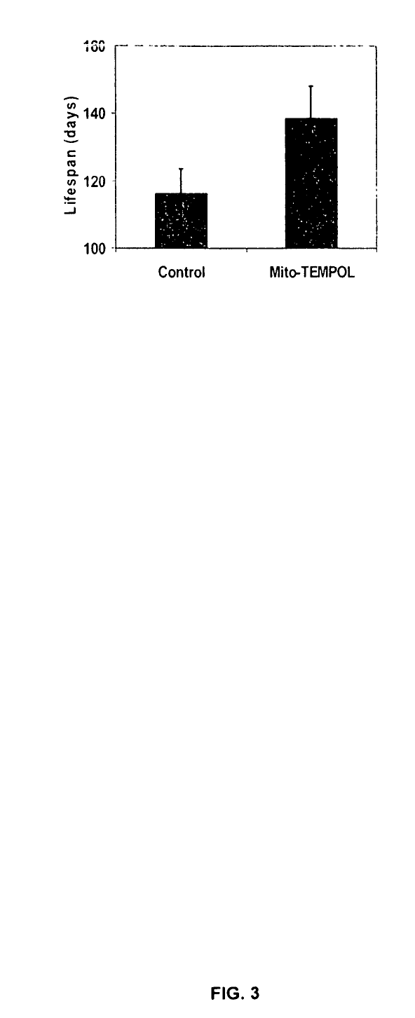

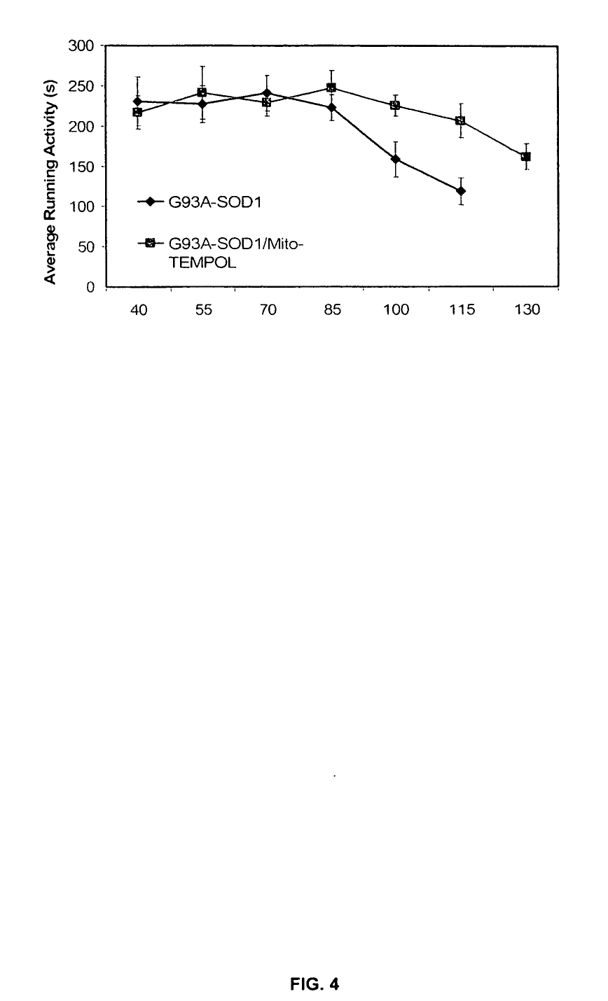

Results: Mito-Tempol treatments delayed disease progression (FIG. 2) and increased the lifespan (FIG. 3) of G93A mice. More significantly, Mito-Tempol treatments promoted the running activity as measured by Rotarod (FIG. 4). Clearly, Mito-Tempol treatment had a marked beneficial effect in promoting motor function, delaying disease progression and extending the lifespan of G93A mice. FIG. 4 likewise shows the gripping strength on a rotarod of G93A mouse (control fed, 105 days old), Tempol-treated G93A mouse (115 days old), and Mito-Tempol-treated G93A mouse (115 days old). Clearly, the beneficial effects of Mito-Tempol are dramatic.

In summary, the newly-synthesized, positively-charged, nitroxide ether, Mito-Tempol, affords neuroprotection in an ALS mouse model. This is the first time a mitochondria-targeted nitroxide or any mitochondria-targeted drug has been used as a therapeutic agent in an ALS animal model.

### Example 3

### Comparison of Mito-Tempol and Tempol

Methods: Mito-Tempol was prepared as described above.

Cell line: Bovine aortic endothelial cells (BAECs; American Type Culture Collection) were grown to confluence in DMEM (Calbiochem; San Diego, Calif.) containing 10% fetal bovine serum (FBS; Invitrogen; Carlsbad, Calif.) insulin (10 μg/ml; Invitrogen), transferrin (5 μg/ml; Invitrogen), glutamine (4 mM; Sigma-Aldrich), penicillin (100 units/ml; Sigma-Aldrich) and streptomycin (100 μg/ml; Sigma-Aldrich) and were incubated at 37° C. in a humidified atmosphere of 5% CO₂ and 95% air. The BAECs were used between passages four and twelve, as described by Balla G, et al., “Ferritin: a cytoprotective antioxidant strategem of endothelium,” J. Biol. Chem. 267:18148-18153 (1992). On the day of treatment, the medium was replaced with DMEM containing 2% FBS.

Glucose/Glucose Oxidase (Glu/GO): GO was obtained from Sigma Aldrich (Milwaukee, Wis.); 13-hydroperoxyoctadecadienoic acid (13-HpODE, a lipoxygenase-catalyzed oxidative metabolite of linolieic acid) and β-hydroxyoctadecadienoic acid (13-HODE, a lipid hydroperoxide) were obtained from Cayman Chemical Co. (Ann Arbor, Mich.); 2′,7′-dichlorodihydrofluorescein diacetate (DCFH-DA) was obtained from Molecular Probes (Eugene, Oreg.); N-carbobenzoxyl-L-leucinyl-L-norleucinal (MG-132) was obtained from Biomol (Plymouth Meeting, Pa.); and clastolactacystin-α-lactone (Lac) was obtained from Sigma Aldrich.

BAECs were incubated with Glu/GO (20 μM), 13-HpODE (25 μM) or 13-HODE (25 μM) for different time periods (0, 2, 4, 8 or 16 hours).

Caspase-3 Activity: After treatment with Glu-GO and antioxidants, cells were washed twice with Dulbecco's phosphate-buffered saline (DPBS) and lysed with cell lysis buffer. Caspase-3 activity was measured with acetyl Asp-Glu-Val-Asp 7-amido-4-methylcoumarin (Ac-DEVD-AFC), as described in a kit from Sigma Aldrich. Caspase-3 hydrolyzes Ac-DEVD-AFC to release a fluorescent moiety, 7-amino-4-methylcoumarin (AMC). Briefly, treated cells were suspended in 100 μl of lysis buffer and passed through a 24-guage needle 10 times to ensure complete lysis. The resulting lysate was centrifuged at 4° C. at 10,000 rmp. 50 μl of clear supernatant was used for the activity assay. An increase in fluorescence was considered as an index of caspase activity, suggesting increased apoptosis. The excitation and emission wavelengths of AMC are 360 nm and 460 nm respectively.

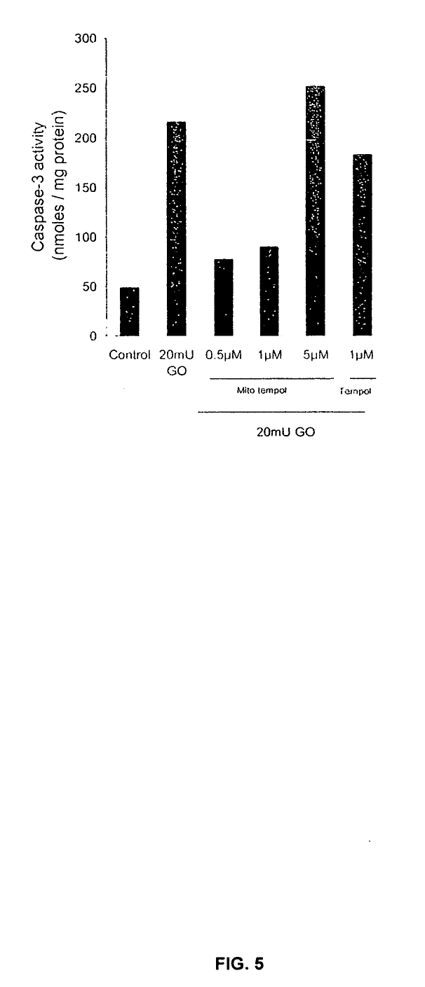

Results: Referring to FIG. 5, Mito-Tempol is more potent than Tempol in inhibiting peroxide-induced apoptosis. BAECs were incubated with Glu/GO for eight hours, as described by Dhanasekaran et al., supra., and monitored for Glu-GO-induced caspase-3 activity as a markers of mitochondria-mediated apoptotic cell death. As shown in FIG. 5, there was a 4-fold increase in caspase-3 activity in control cells and Tempol-treated cells compared to Mito-Tempol-treated cells. Pretreatment of BAECs with Mito-TEMPOL (0.5 and 1 μM) greatly inhibited Glue/GO-mediated caspase-3 activation. In contrast, Tempol did not inhibit the caspase-3 activation even at a 10-fold higher concentration (1 and 5 μM). This result suggests that Mito-Tempol is more potent than Tempol in mitigating ROS-induced apoptosis in cells.

The present invention has been described in connection with what are presently considered to be the most practical and preferred embodiments. However, the present invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

We claim:

1. A method of treating amyotrophic lateral sclerosis, comprising the step of administering to a subject having or at risk of having amyotrophic lateral sclerosis an effective amount of a mitochondria-targeted nitroxide selected from a positively charged nitroxide ether, a positively charged nitroxide ester and a positively charged nitroxide amide, such that the symptoms of amyotrophic lateral sclerosis are attenuated.

2. A method as recited in claim 1, wherein the mitochondria-targeted nitroxide is selected from Mito-Tempol ether, Mito-Tempol ester, Mito-Tempol amide, Mito-CP, Mito-Tempone, Mito-Tempamide, Mito-Proxyl ether, Mito-Proxyl ester, Mito-Proxyl amide, Tributylalkylammonium Tempol ether and Tribenzyalkylammonium Tempol ether.

3. A method as recited in claim 1, wherein the mitochondria-targeted nitroxide is Mito-Tempol ether.

4. A method as recited in claim 1, wherein the mitochondria-targeted nitroxide is Mito-Tempol ester.

5. A method as recited in claim 1, wherein the mitochondria-targeted nitroxide is administered orally.

6. A method as recited in claim 1, wherein the mitochondria-targeted nitroxide is administered intravenously.

7. A method as recited in claim 1, wherein the mitochondria-targeted nitroxide is administered intraperitoneally.

8. A method as recited in claim 1, wherein the symptoms of amyotrophic lateral sclerosis are attenuated by at least about 10%.

9. A method as recited in claim 1, wherein the symptoms of amyotrophic lateral sclerosis are attenuated by at least about 15%.

10. A method as recited in claims 1, wherein the symptoms of amyotrophic lateral sclerosis are attenuated by at least about 20%.

11. A method as recited in claim 1, wherein the symptoms of amyotrophic lateral sclerosis are attenuated by at least about 25%.

12. A method as recited in claim 1, wherein the symptoms of amyotrophic lateral sclerosis are selected from impaired motor neuron function, mitochondria dysfunction, increased reactive oxygen species, oxidative damage, protein nitration, aggregation of proteins and decreased cell survival.

13. A method as recited in claim 1, wherein the mitochondria-targeted nitroxide is administered in water.

14. A method as recited in claim 1, wherein the amount of mitochondria-targeted nitroxide administered is between about 30 mg/kg to about 60 mg/kg body weight.

15. A method of attenuating mitochondria dysfunction comprising the step of administering to a subject at having or at risk of having reactive oxygen species-mediated mitochondria dysfunction an effective amount of a mitochondria-targeted nitroxide selected from a positively charged nitroxide ether, a positively charged nitroxide ester and a positively charged nitroxide amide, such that the apoptosis is attenuated.

16. A method as recited in claim 15, wherein the mitochondria-targeted nitroxide is selected from Mito-Tempol ether, Mito-Tempol ester, Mito-Tempol amide, Mito-CP, Mito-Tempone, Mito-Tempamide, Mito-Proxyl ether, Mito-Proxyl ester, Mito-Proxyl amide, Tributylalkylammonium Tempol ether and Tribenzyalkylammonium Tempol ether.

17. A method as recited in claim 15, wherein the mitochondria-targeted nitroxide is Mito-Tempol ether.

18. A method as recited in claim 15, wherein the mitochondria-targeted nitroxide is Mito-Tempol ester.

19. A method as recited in claim 15, wherein the reactive oxygen species-mediated mitochondria dysfunction is caused by a disorder selected from diabetes, hypertension, ischemia and reperfusion injury, and radiation-induced mitochondria dysfunction.

20. Mito-Tempol prepared by a method comprising the steps of;
(a). obtaining a sodium derivative of Tempol;
(b). obtaining a bromobutyl ether of Tempol from the sodium derivative of Tempol;
(c). combining the bromobutyl ether of Tempol with triphenylphosphine to obtain Mito-Tempol; and
(d). analyzing purity of the Mito-Tempol with electron paramagnetic resonance and liquid chromatography-mass spectrometry such that the purity is at least 90%.

21. Mito-Tempol prepared by a method as recited in claim 20, wherein the purity is at least 95%.

22. Mito-Tempol prepared by a method as recited in claim 20, wherein the purity is at least 99%.

23. Mito-Tempol prepared by a method as recited in claim 20, wherein the sodium derivative of Tempol is prepared by mixing a stoichiometric amount of sodium hydride with Tempol.

24. Mito-Tempol prepared by a method as recited in claim 23, wherein the stoichiometric amount is about 1:1 to about 1:2.

25. Mito-Tempol prepared by a method as recited in claim 20, wherein benzene is used as a solvent.
